(19) United States
(12) Patent Application Publication
(10) Pub. No.: US 2025/0364087 A1
(43) Pub. Date: Nov. 27, 2025

(54) QUANTUM CHEMISTRY COMPUTATION METHOD AND INFORMATION PROCESSING APPARATUS

(71) Applicant: Fujitsu Limited, Kawasaki-shi (JP)

(72) Inventor: Eiji OHTA, Yokohama (JP)

(73) Assignee: Fujitsu Limited, Kawasaki-shi (JP)

(21) Appl. No.: 19/296,283

(22) Filed: Aug. 11, 2025

Related U.S. Application Data
(63) Continuation of application No. PCT/JP2023/045657, filed on Dec. 20, 2023.

(30) Foreign Application Priority Data
Feb. 15, 2023 (JP) 2023-021304

(51) Int. Cl. G16C 10/00

(52) U.S. Cl. CPC G16C 10/00

(57) ABSTRACT

An information processing apparatus iteratively calculates, using a self-consistent field method, an electron density at each of a plurality of points in a space where a substance exists. The information processing apparatus determines, for each of the plurality of points, each time the electron density is calculated, whether a plurality of indicator values based on the calculated electron density satisfies respective convergence criteria respectively associated with the plurality of indicator values. Then, the information processing apparatus terminates, for each of the plurality of points, the iteratively calculating of the electron density upon at least one of the plurality of indicator values satisfying the associated convergence criterion.

### CROSS-REFERENCE TO RELATED APPLICATIONS

This application is a continuation application of International Application PCT/JP2023/045657 filed on Dec. 20, 2023, which designated the U.S., which is based upon and claims the benefit of priority of the prior Japanese Patent Application No. 2023-021304, filed on Feb. 15, 2023, the entire contents of which are incorporated herein by reference.

### FIELD

The embodiments discussed herein relate to a quantum chemistry computation method and an information processing apparatus.

### BACKGROUND

In recent years, materials informatics (hereinafter referred to as “MI”) has advanced globally in the field of materials development as a data-driven research and development approach, with the aim of shortening research and development periods and reducing costs. In MI, the accumulation of high-quality material data is important. Therefore, in addition to experiments, efficient data accumulation using simulations such as quantum chemistry computation is expected.

One of the major computation methods in quantum chemistry is density functional theory (DFT). In DFT, the electron density of a target substance is first calculated by a self-consistent field (SCF) method. Then, based on the obtained electron density, physical quantities indicating the state of the substance (such as total energy) calculated.

As a technique related to DFT, an electronic state computation method has been proposed in which, starting from an existing approximation model, a plurality of computational pathways are explored within a model space in accordance with the variational principle of density functional theory, with the aim of reaching the physical properties indicated by the exact solution through a finite number of computations.

See, for example, International Publication Pamphlet No. WO 2012/023563.

### SUMMARY

In one aspect, there is provided a non-transitory computer-readable recording medium storing therein a computer program that causes a computer to execute a process including: iteratively calculating, using a self-consistent field method, an electron density at each of a plurality of points in a space where a substance exists; determining, for each of the plurality of points, each time the electron density is calculated, whether a plurality of indicator values based on the calculated electron density satisfies respective convergence criteria respectively associated with the plurality of indicator values; and terminating, for each of the plurality of points, the iteratively calculating of the electron density upon at least one of the plurality of indicator values satisfying the associated convergence criterion.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWING

FIG. 1 illustrates an example of a quantum chemistry computation method according to a first embodiment;

FIG. 2 illustrates example of a system configuration;

FIG. 3 illustrates an example of hardware of a control node;

FIG. 4 illustrates an example of a hardware configuration of computing nodes;

FIG. 5 illustrates a relationship between the magnitude of electron density and a number of SCF iterations until a difference converges;

FIG. 6 illustrates an example of electron density differences for each SCF iteration under different electron density magnitudes;

FIG. 7 illustrates, for points located in subspaces with low electron density, how the difference and a difference change rate vary with the number of SCF iterations;

FIG. 8 illustrates an example of convergence determination for each subspace based on the electron density;

FIG. 9 is a block diagram illustrating an example of the functions implemented by a computing system;

FIG. 10 is a flowchart illustrating an example of a procedure for an energy computation process;

FIG. 11 illustrates an example of subspaces;

FIG. 12 illustrates an example of subspaces set based on electron densities;

FIG. 13 is a flowchart illustrating an example of a procedure for a subspace setting process;

FIG. 14 illustrates an example of allowable value information;

FIG. 15 is a flowchart illustrating an example of a procedure for a SCF calculation process;

FIG. 16 illustrates an example of an acceleration factor;

FIG. 17 illustrates an example of acceleration of SCF calculation;

FIG. 18 illustrates the acceleration factor when both the difference and the difference change rate of the electron density are used as convergence criteria; and

FIG. 19 illustrates an example of a wide-range exploration service.

### DESCRIPTION OF EMBODIMENTS

Assuming that the number of atoms is N (where N is a natural number), the computational cost of DFT scales as O(N³). Consequently, as the size of the target system increases, the computational cost becomes enormous. One of the factors contributing to this high computational cost is the large number of iterations involved in the electron density calculation performed by a SCF method.

In the SCF method, the electron density at each of a plurality of points (coordinates) defined within a spatial region under analysis is computed iteratively. The SCF calculation is regarded as converged when convergence criteria for the electron density are satisfied at all of the points. Therefore, if the convergence condition is not met at even a portion of the points, the SCF calculation does not converge, resulting in extended computation time.

Hereinafter, embodiments will be described with reference to the accompanying drawings. It should be noted that, as long as no inconsistencies arise, the respective embodiments may be implemented in combination with one another.

### (a) First Embodiment

A first embodiment relates to a quantum chemistry computation method that shortens the computation time of the electron density by reducing the number of iterations in the SCF calculation (hereinafter simply referred to as the number of SCF iterations) that are involved in determining the electron density of a substance.

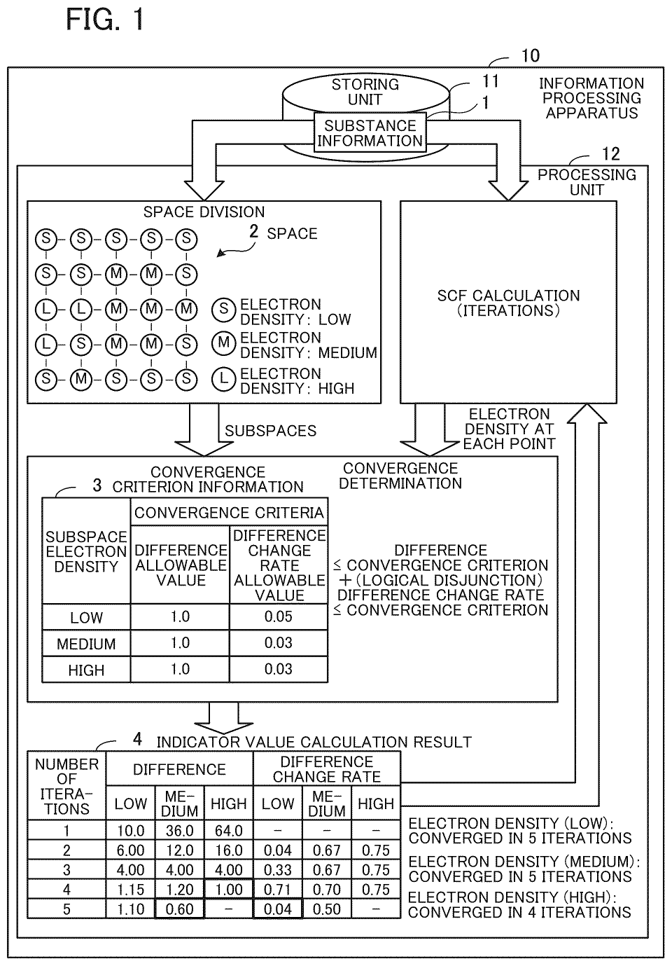

FIG. 1 illustrates an example of the quantum chemistry computation method according to the first embodiment. In FIG. 1, an information processing apparatus 10 for performing the quantum chemistry computation method is depicted. The information processing apparatus 10 may, for example, implement the quantum chemistry computation method by executing a quantum chemistry computation program.

The information processing apparatus 10 includes a storing unit 11 and a processing unit 12. The storing unit 11 may be, for example, a memory or storage device included in the information processing apparatus 10. The processing unit 12 may be, for example, a processor or arithmetic circuit included in the information processing apparatus 10.

The storing unit 11 stores substance information 1, which indicates a substance to be analyzed. The substance information 1 includes, for example, information such as atoms contained in the substance, bonding conditions between atoms, and interatomic distances. The substance information 1 further includes, for example, initial values of electron densities at a plurality of points within a space 2 in which the substance is present.

The processing unit 12 calculates the electron density of the substance to be analyzed in its ground state (the state of minimum energy) using DFT, based on the substance information 1. For example, the processing unit 12 iteratively calculates the electron density at each of a plurality of points within the space 2 in which the substance is present, using a SCF method. Each time the electron density is calculated, the processing unit 12 determines, for each of the plurality of points, whether a plurality of indicator values based on the calculated electron density satisfy their respective convergence criteria.

The plurality of indicator values include, for example, the difference between the previous and current calculation results of the electron density, and the difference change rate, which indicates the degree of change between the previous and current calculation results of the difference of the electron density. A convergence criterion for the difference may be represented by a difference allowable value. If the difference of the electron density at a point to be evaluated is equal to or less than the difference allowable value, the electron density at that point is determined to satisfy the convergence criterion for the difference. A convergence criterion for the difference change rate may be represented by a difference change rate allowable value. If the difference change rate of the electron density at the point to be evaluated is equal to or less than the difference change rate allowable value, the electron density at that point is determined to satisfy the convergence criterion for the difference change rate.

Then, for each point, if at least one of the plurality of indicator values satisfies its respective convergence criterion, the processing unit 12 terminates the iterative calculation of the electron density. That is, the convergence of the electron density at each point is determined based on the logical disjunction of the convergence determinations of the respective indicator values at that point.

For example, if the plurality of indicator values are the difference and the difference change rate of the electron density, the processing unit 12 determines that the overall electron density has converged and terminates the iterative calculation of the electron density, provided that at least one of the difference and the difference change rate satisfies its convergence criterion at each point.

In this manner, the processing unit 12 terminates the SCF calculation by determining that the overall electron density has converged based on at least one of the plurality of indicator values at each point satisfying its convergence criterion. In other words, even if the difference of the electron density at a point to be evaluated does not satisfy its convergence criterion, if the difference change rate does, the electron density at that point is considered to have converged. As a result, compared to a case in which only the difference is used for determining convergence, the number of SCF iterations is reduced. The reduction in the number of SCF iterations shortens the computation time of the electron density.

For example, at points where the electron density is relatively low, the electron density repeatedly increases and decreases, making it difficult for the difference of the electron density to satisfy the convergence criterion for the difference. Even in the presence of such points, the SCF calculation is terminated if the difference change rate at those points satisfies the convergence criterion for the difference change rate. Notably, even if the electron density continues to fluctuate at points with low electron density, its effect on the total energy of the substance to be analyzed is minimal. Therefore, even if the calculation of the electron density is terminated before the difference of the electron density satisfies the convergence criterion at such points, the impact on the total energy calculated based on that electron density is negligible. As a result, deterioration in computational accuracy is minimized.

The processing unit 12 may divide the space 2 to be analyzed into a plurality of subspaces in advance based on the initial values of the electron densities. For example, the processing unit 12 may classify each of the plurality of points into one of the plurality of subspaces based on the initial values of the electron densities of the plurality of points. In this case, the processing unit 12 calculates a plurality of first indicator values for a first target point belonging to a first subspace among the classified subspaces. Then, for each of the calculated first indicator values, the processing unit 12 determines whether the first target point satisfies the convergence criterion set for the first subspace to which the first target point belongs. For each subspace, a convergence criterion is set for each of the indicator values.

In this way, by dividing the space 2 into subspaces and setting convergence criteria for each subspace, the processing unit 12 is able to, for example, set looser convergence criteria for subspaces including points with low impact on the total energy.

In the example depicted in FIG. 1, the space 2 is divided into three subspaces based on the electron densities. The first subspace includes points with low electron density (S-points in the space 2). The second subspace includes points with medium electron density (M-points in the space 2). The third subspace includes points with high electron density (L-points in the space 2).

The convergence criteria for each subspace are presented in convergence criterion information 3. In the convergence criterion information 3 illustrated in FIG. 1, the convergence criterion for the difference (difference allowable value) is the same across all the subspaces. On the other hand, the convergence criterion for the difference change rate (difference change rate allowable value) is set to a larger value for the subspace including points with low electron density than for other subspaces. This reduces the number of SCF iterations and shortens the computation time.

For example, an indicator value calculation result 4 presents a transition example of the difference and the difference change rate for each subspace in each SCF iteration. In the example of the indicator value calculation result 4, the difference at points within the subspace including points with low electron density is greater than the difference allowable value (i.e., the convergence criterion is not satisfied) even at the fifth SCF iteration. On the other hand, the difference change rate calculated in the fifth SCF iteration is equal to or less than the difference change rate allowable value (the convergence criterion is satisfied). Accordingly, the electron density within the subspace including points with low electron density converges through five SCF iterations.

The difference change rate at points within the subspace including points with medium electron density is greater than the difference change rate allowable value (i.e., the convergence criterion is not satisfied) even at the fifth SCF iteration. On the other hand, the difference calculated in the fifth SCF iteration is equal to or less than the difference allowable value (the convergence criterion is satisfied). Accordingly, the electron density within the subspace including points with medium electron density converges through five SCF iterations.

The difference change rate at points within the subspace including points with high electron density is greater than the difference change rate allowable value (i.e., the convergence criterion is not satisfied) even at the fourth SCF iteration. On the other hand, the difference calculated in the fourth SCF iteration is equal to or less than the difference allowable value (the convergence criterion is satisfied). Accordingly, the electron density within the subspace including points with high electron density converges through four SCF iterations.

In this manner, by setting appropriate convergence criteria for the difference and the difference change rate for each subspace, the processing unit 12 is capable of performing convergence determination while taking into account subspace-dependent variations in the behavior of the difference or the difference change rate with respect to the number of SCF iterations. As a result, the number of SCF iterations is reduced.

When the space 2 is divided into a plurality of subspaces, the processing unit 12 may also determine, for each subspace, whether convergence has been achieved. For example, the processing unit 12 determines that a first subspace, among the plurality of subspaces, has converged if, for each point belonging to the first subspace, at least one of the plurality of indicator values satisfies the convergence criterion. Thereafter, the processing unit 12 determines whether each point belonging to a second subspace, which is different from the first subspace determined to have converged, satisfies the respective convergence criteria. As a result, for points in converged subspaces, it is no longer needed to calculate indicator values (such as the difference and the difference change rate) and to compare them with the convergence criteria, thereby enabling convergence determination to be performed efficiently.

### (b) Second Embodiment

A second embodiment relates to a computing system configured to reduce the SCF calculation time in performing a molecular state calculation of a substance using DFT on high-performance computing (HPC).

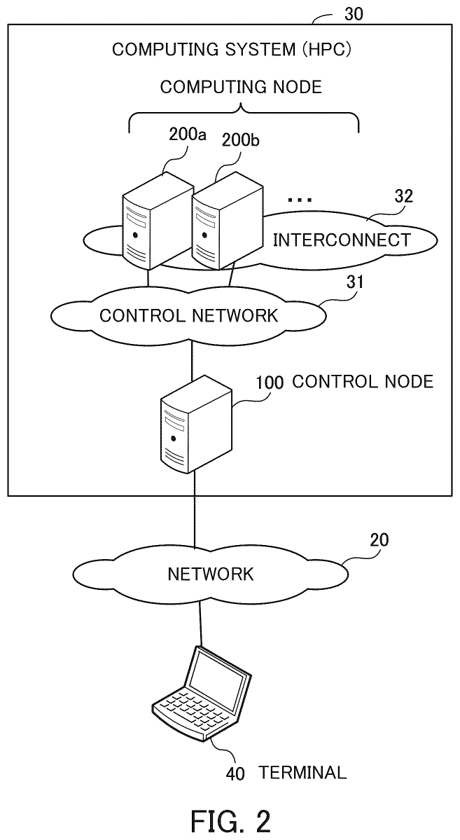

FIG. 2 illustrates an example of a system configuration. A computing system 30 that realizes HPC includes a control node 100 and a plurality of computing nodes 200a, 200b, and the like. The plurality of computing nodes 200a, 200b, and the like is connected to one another via an interconnect 32 that enables high-speed communication among the nodes. The interconnect 32 may be, for example, a network in which processors are interconnected via a six-dimensional mesh or torus topology.

Each of the computing nodes 200a, 200b, and the like is also connected to a control network 31. The control network 31 is further connected to the control node 100. The control node 100 is a computer configured to issue job execution instructions to the computing nodes 200a, 200b, and the like.

The control node 100 is connected to a terminal 40 via a network 20. The terminal 40 is a computer that, based on user operations, registers job information, specifying jobs to be executed by the computing nodes 200a, 200b, and the like, with the control node 100. In accordance with instructions from the terminal 40, the control node 100 instructs the computing nodes 200a, 200b, and the like to perform computation based on DFT.

For example, by causing the computing nodes 200a, 200b, and the like to execute a calculation of the state of a substance using DFT, the user obtains physical quantities such as the total energy of an electronic system defined by DFT.

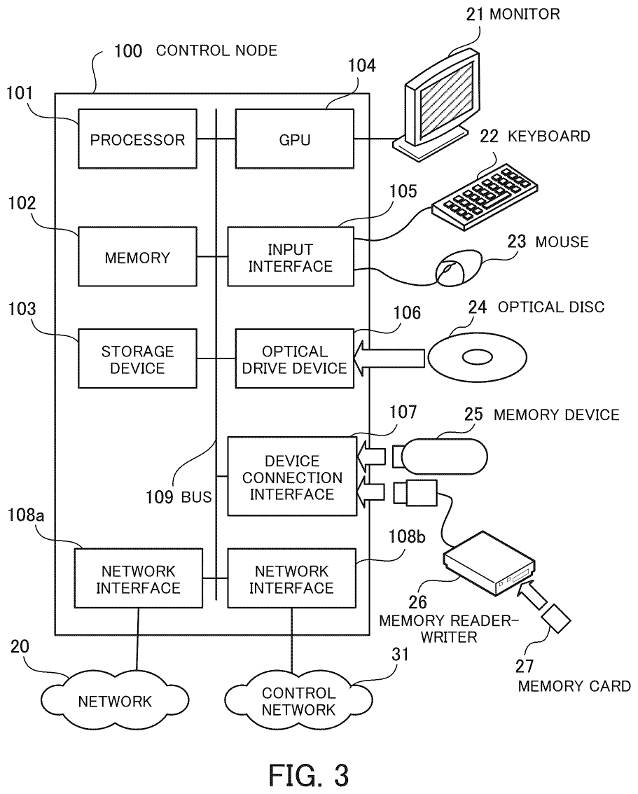

FIG. 3 illustrates an example of hardware of the control node. The control node 100 is controlled entirely by a processor 101. The processor 101 is connected via a bus 109 to a memory 102 and to a plurality of peripheral devices. The control node 100 may be a multiprocessor system that includes a plurality of processors. A set of processors in the multiprocessor system may be referred to as the processor 101. The processor 101 may also be referred to as processor circuitry. Each of the plurality of processors may execute a part or all of the processes executed by the control node 100. When there is a plurality of related processes, two or more of the processes may be executed by different processors. The processor 101 may be, for example, a central processing unit (CPU), a micro processing unit (MPU), or a digital signal processor (DSP). At least a part of the functions realized by the processor 101 through program execution may alternatively be implemented by electronic circuits such as an application specific integrated circuit (ASIC) or programmable logic device (PLD).

The memory 102 is used as a main storage device of the control node 100. The memory 102 temporarily stores at least part of an operating system (OS) program and application programs to be executed by the processor 101. The memory 102 also stores various types of data used for processing by the processor 101. For example, the memory 102 may be a volatile semiconductor storage device such as a random access memory (RAM).

The peripheral devices connected to the bus 109 include a storage device 103, a graphics processing unit (GPU) 104, an input interface 105, an optical drive device 106, a device connection interface 107, and network interfaces 108a and 108b.

The storage device 103 performs electrical or magnetic writing and reading of data to and from a built-in recording medium. The storage device 103 is used as an auxiliary storage device of the control node 100. The storage device 103 stores programs for the OS, application programs, and various types of data. For example, the storage device 103 may be a hard disk drive (HDD) or a solid state drive (SSD).

The GPU 104 is a processing unit for image processing and is also referred to as a graphics controller. A monitor 21 is connected to the GPU 104. The GPU 104 displays images on the screen of the monitor 21 in accordance with instructions from the processor 101. Examples of the monitor 21 include an organic electro luminescence (EL) display and a liquid crystal display.

A keyboard 22 and a mouse 23 are connected to the input interface 105. The input interface 105 transmits signals received from the keyboard 22 and the mouse 23 to the processor 101. The mouse 23 is an example of a pointing device, and other pointing devices may be used instead. Examples of other pointing devices include a touch panel, a tablet, a touchpad, and a trackball.

The optical drive device 106 performs reading of data recorded on an optical disc 24 or writing of data to the optical disc 24 using laser light or the like. The optical disc 24 is a portable recording medium on which data is recorded to be readable by light reflection. Examples of the optical disc 24 include a digital versatile disc (DVD), DVD-RAM, compact disc read only memory (CD-ROM), CD-recordable (CD-R), and CD-rewritable (CD-RW).

The device connection interface 107 is a communication interface for connecting peripheral devices to the control node 100. For example, a memory device 25 and a memory reader-writer 26 may be connected to the device connection interface 107. The memory device 25 is a recording medium equipped with a communication function with the device connection interface 107. The memory reader-writer 26 is a device that writes data to or reads data from a memory card 27. The memory card 27 is a card-type recording medium.

The network interface 108a is connected to the network 20. The network interface 108a transmits and receives data to and from other computers or communication devices such as the terminal 40 via the network 20. The network interface 108b is connected to the control network 31. The network interface 108b transmits and receives data to and from the computing nodes 200a, 200b, and the like via the control network 31.

The network interfaces 108a and 108b are wired communication interfaces that are connected by cables to wired communication devices such as switches or routers. However, the network interfaces 108a and 108b may instead be implemented as wireless communication interfaces that are connected by radio waves to wireless communication devices such as base stations or access points.

The control node 100 realizes the processing functions of the second embodiment by executing a program recorded on a computer-readable recording medium, for example. The program describing the processing to be executed by the control node 100 may be recorded on various recording media. For example, the program to be executed by the control node 100 may be stored in the storage device 103. The processor 101 loads at least a part of the program from the storage device 103 into the memory 102 and executes the program. The program to be executed by the control node 100 may also be recorded on portable recording media such as the optical disc 24, the memory device 25, or the memory card 27. The program stored on portable recording media may be installed into the storage device 103 under the control of the processor 101 and then executed. Alternatively, the processor 101 may directly read and execute the program from portable recording media.

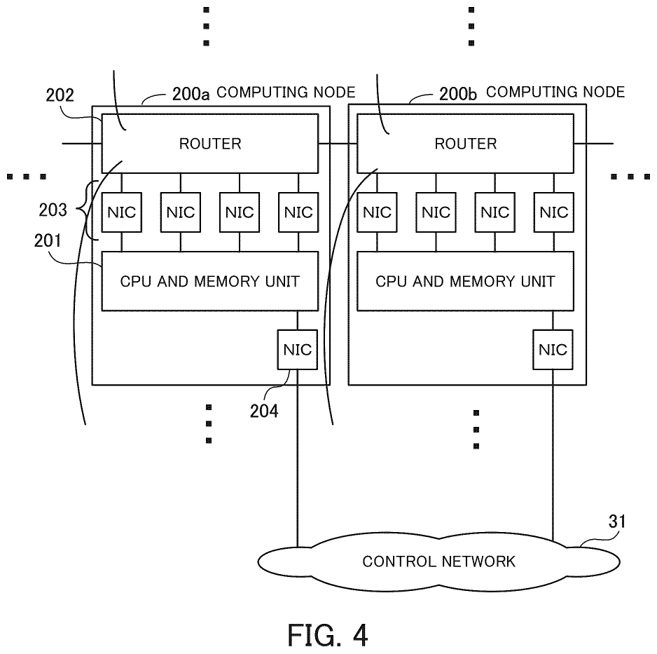

FIG. 4 illustrates an example of a hardware configuration of the computing nodes. The computing node 200a includes a CPU and memory unit 201 and a router 202. The CPU and memory unit 201 and the router 202 are connected via a plurality of communication interfaces (NICs) 203. In addition, the CPU and memory unit 201 is also connected to a NIC 204 for connection to the control network 31.

The CPU and memory unit 201 includes a CPU having a plurality of cores and a memory. A process (a unit of execution) is generated for each core in the CPU and memory unit 201. When a process on a core of the CPU and memory unit 201 performs synchronization processing with processes in other computing nodes, such as the computing node 200b, communication is conducted via the router 202 with the other computing nodes.

The router 202 communicates with adjacent computing nodes in, for example, each of the three-dimensional directions. The router 202 transmits data from the CPU and memory unit 201 to an adjacent computing node in the direction corresponding to the position of destination computing node within the interconnect 32. Upon receiving data destined for a process in the CPU and memory unit 201 from an adjacent computing node, the router 202 transfers the data to the CPU and memory unit 201. Further, when data received from an adjacent computing node is destined for a different computing node, the router 202 forwards the data to an adjacent computing node in the direction corresponding to the position of the destination node within the interconnect 32.

The other computing nodes 200b and the like have the same hardware configuration as the computing node 200a. The interconnect 32 is a three-dimensional mesh or torus interconnect formed by connecting the computing nodes 200a, 200b, and the like having the hardware configuration depicted in FIG. 4 in a mesh or torus topology along three-dimensional directions.

The processing functions of the second embodiment are implemented by the control node 100 and the computing nodes 200a, 200b, and the like having the above-described hardware configurations. It is noted that the information processing apparatus 10 illustrated in the first embodiment may also be implemented using the same hardware as the control node 100 or the computing node 200a.

In the computing system 30, DFT is used to calculate physical quantities representing the state of a substance, such as total energy. In DFT, the electron density is calculated using the SCF method. In the electron density calculation by the SCF method, the calculation is iteratively performed until the electron density at all points arranged in a space or coordinate system has converged. As a result, the self-consistent total energy is obtained.

Here, the causes of increased iterations are described for the repeated electron density calculation using the SCF method, in cases where the electron density does not converge. As a convergence criterion for determining whether the electron density at each point has converged, for example, the difference from the previous value of the electron density at the corresponding point may be used. The convergence criterion for determining whether the electron density at an i-th point rᵢ (where i is a natural number) has converged may be expressed, for example, by the following expression: Δρ(rᵢ)=|ρ(rᵢ)−ρ′(rᵢ)|≤allowable value of Δρ.

Δρ(rᵢ) represents the difference of the electron density at the i-th point. ρ′(rᵢ) is the electron density at the i-th point obtained from the latest SCF iteration. ρ(rᵢ) is the electron density at the i-th point obtained from the previous SCF iteration. The allowable value of Δρ is a preset value.

In the SCF calculation, convergence of the overall electron density is determined based on whether the convergence criterion is satisfied at all points. However, if there are any points (referred to as difficult-to-converge points) that fall into any of the following cases below, the total electron energy may exhibit slight fluctuations, and the SCF convergence condition (i.e., the convergence of the overall electron density) may remain unfulfilled.

As a first type of difficult-to-converge point, there are points where the effect on the electron energy is small (i.e., the electron density is low), but the electron density repeatedly increases and decreases, and the convergence criterion remains unsatisfied.

As a second type of difficult-to-converge point, there are points where the change rate of the difference (the difference change rate) is small, but the difference continues to vary, and the convergence criterion remains unsatisfied.

When such difficult-to-converge points exist, it becomes difficult to determine that the overall electron density has converged, and the number of SCF iterations increases. These difficult-to-converge points are often located in regions of relatively low electron density. The approximate electron density at any point in a substance may be inferred from the atomic arrangement and bonding state. For example, the electron density at an atomic position is high. The electron density at bonding regions is of intermediate magnitude. The electron density in regions representing molecular shapes is low.

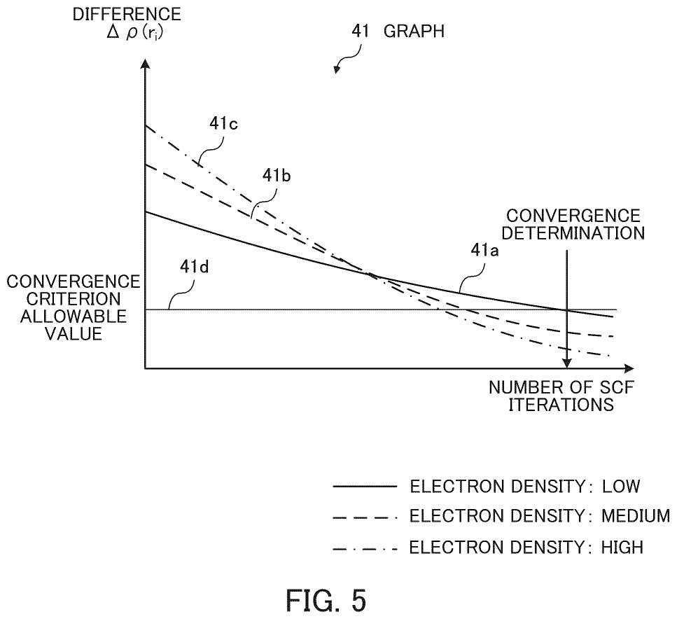

FIG. 5 illustrates the relationship between the magnitude of electron density and the number of SCF iterations until the difference converges. In a graph 41 depicted in FIG. 5, the horizontal axis represents the number of SCF iterations, and the vertical axis represents the difference of the electron density at each SCF iteration. A solid curve 41a in the graph 41 indicates the change in the electron density difference at points in regions of low electron density. A dashed curve 41b indicates the change in the electron density difference at points in regions of medium electron density. A dash-dot curve 41c indicates the change in the electron density difference at points in regions of high electron density. A horizontal line 41d in the graph 41 indicates an allowable value of the convergence criterion.

As represented in the graph 41, points in regions of high electron density satisfy the convergence criterion at an early stage. On the other hand, points in regions of low electron density take more SCF iterations to satisfy the convergence criterion.

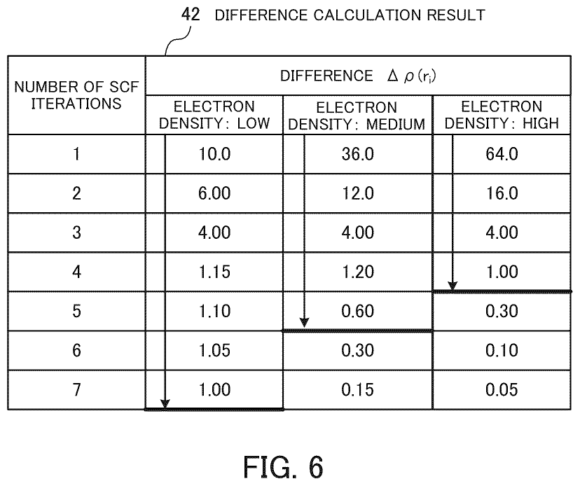

FIG. 6 illustrates an example of electron density differences for each SCF iteration under different electron density magnitudes. In a difference calculation result 42 illustrated in FIG. 6, the electron density difference per SCF iteration is depicted for points classified by the magnitude of the electron density. Here, the allowable value Δρ for the electron density difference is assumed to be 1.0 (Δρ=1.0).

In the example of the difference calculation result 42, points in regions of low electron density satisfy the convergence criterion at the seventh SCF iteration. Points in regions of medium electron density satisfy the convergence criterion at the fifth iteration. Points in regions of high electron density satisfy the convergence criterion at the fourth iteration. As a result, it takes seven SCF iterations for the electron density over the entire space to converge.

To address the above, the computing system 30 promotes earlier convergence for the above-mentioned difficult-to-converge points without degrading computational accuracy. For example, the computing system 30 divides the space subject to the convergence determination in the SCF iterative calculation into a plurality of subspaces. Next, the computing system 30 calculates the difference change rate of the electron density for each subregion. The computing system 30 then sets respective convergence criteria for the difference and the difference change rate for each subregion. Then, the computing system 30 determines that the SCF calculation has converged when, in all subspaces, at least one of the electron density difference and the difference change rate satisfies the convergence criterion for all points belonging to each subspace.

By dividing the space into a plurality of subspaces according to the electron density and performing convergence determination for each subspace, this approach reduces the number of SCF iterations until the convergence criterion is satisfied in all subspaces. As a result, the calculation time of the entire DFT is also reduced.

The difference change rate represents the degree of change between the current and previous differences. The difference change rate may be expressed by, for example, the following expression: difference change rate=|(current difference−previous difference)÷previous difference|.

Alternatively, instead of the previous difference, a representative value (such as the average or median) of all differences obtained in past SCF iterations may be used. The convergence criterion for the difference change rate may be set for each subspace. For example, the convergence criterion may be defined as the minimum difference change rate (i.e., allowable value) regarded as indicating convergence. Assuming that the allowable value for the difference change rate in the i-th subspace is denoted as DTHᵢ, the convergence criteria for the difference change rate may be, for example, DTH₁=5%, DTH₂=3%, and DTH₃=3%.

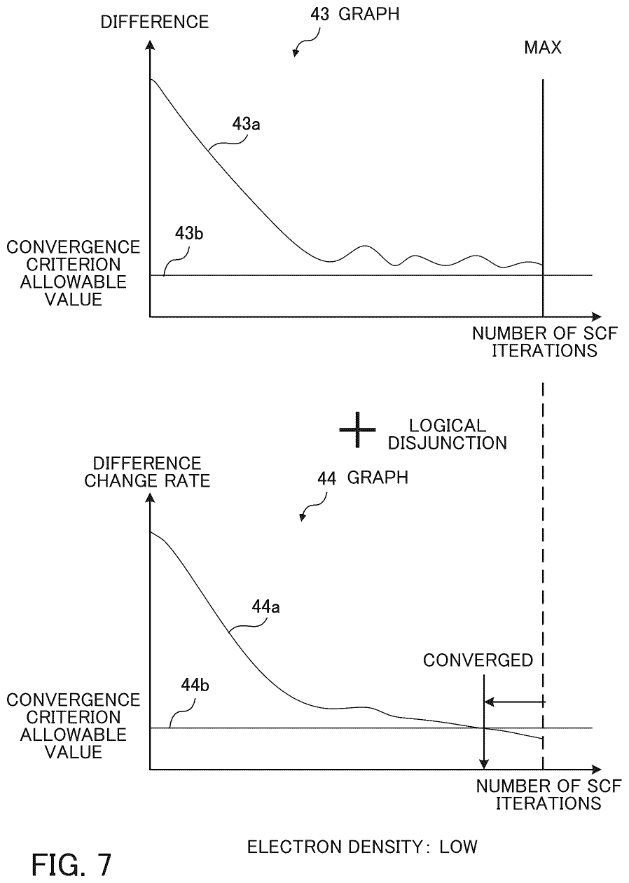

FIG. 7 illustrates, for points located in subspaces with low electron density, how the difference and the difference change rate vary with the number of SCF iterations. A graph 43 depicts the relationship between the electron density difference and the number of SCF iterations. In the graph 43, the horizontal axis represents the number of SCF iterations, and the vertical axis represents the difference. A curve 43a represents the change in the electron density difference at points in subspaces with low electron density. A horizontal line 43b indicates the allowable value for the difference serving as a convergence criterion.

As illustrated in the graph 43, points within subspaces with low electron density exhibit a gradual decrease in the electron density difference during the initial stage of the SCF calculation. However, after the difference has decreased to a certain extent, the electron density difference begins to repeatedly increase and decrease before satisfying the convergence criterion. As a result, even if the SCF calculation is repeated up to the maximum number of iterations, the electron density difference may fail to fall below the convergence criterion. After the point at which the electron density difference begins to vary as depicted in the graph 43, i.e., when the difference starts to fluctuate repeatedly, increased iterations of SCF calculations no longer significantly improve the accuracy of the final total energy.

A graph 44 depicts the relationship between the difference change rate and the number of SCF iterations. In the graph 44, the horizontal axis represents the number of SCF iterations, and the vertical axis represents the difference change rate. A curve 44a represents the change in the electron density difference change rate at points in subspaces with low electron density. A horizontal line 44b indicates the allowable value for the difference change rate serving as a convergence criterion.

As illustrated in the graph 44, the difference change rate at points in subspaces with low electron density decreases as the number of SCF iterations increases. The difference change rate becomes less than or equal to the convergence criterion before the number of SCF iterations reaches its maximum.

In the computing system 30, it is determined whether the electron density at each point has converged, based on a logical disjunction of the convergence determination for the difference and the convergence determination for the difference change rate. Accordingly, the electron density at a given point is determined to have converged if the difference change rate is less than or equal to its convergence criterion, even if the difference does not satisfy its own convergence criterion.

As a result, convergence is determined at an early stage even at points in subspaces with low electron density, thereby reducing the number of SCF iterations.

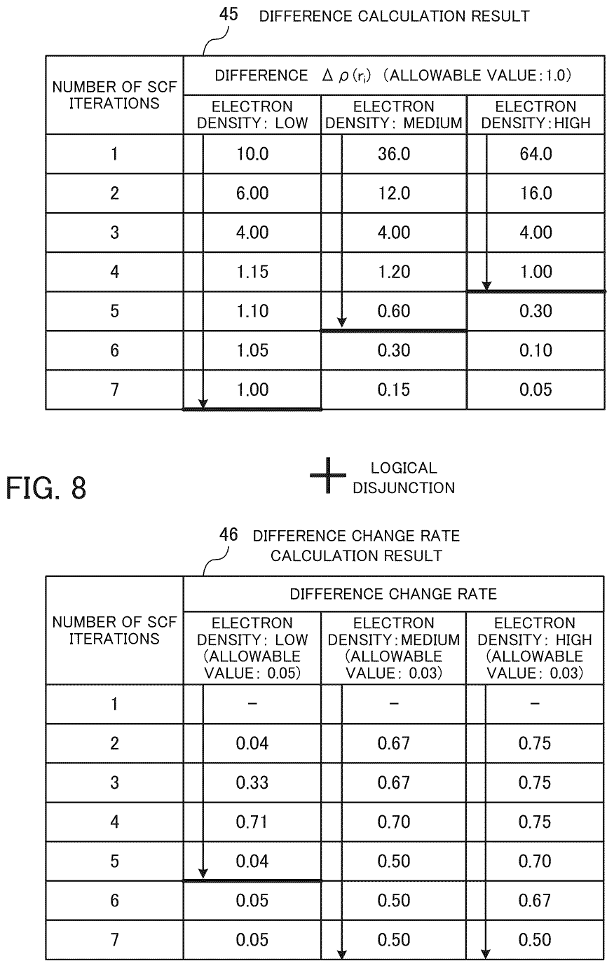

FIG. 8 illustrates an example of convergence determination for each subspace based on the electron density. In a difference calculation result 45, the electron density differences at each SCF iteration are presented according to the magnitude of the electron density at each point. Here, the allowable value Δρ for the electron density difference is assumed to be 1.0.

In a difference change rate calculation result 46, the difference change rates of the electron density at each SCF iteration are presented according to the magnitude of the electron density at each point. The allowable value for the difference change rate is assumed to be 0.05 for subspaces with low electron density, and 0.03 for subspaces with medium and high electron density.

In the example depicted in FIG. 8, the electron density difference at points in subspaces with low electron density becomes less than or equal to the allowable value at the seventh SCF iteration. On the other hand, the difference change rate at the points in subspaces with low electron density becomes less than or equal to the allowable value at the fifth SCF iteration. Therefore, the points in subspaces with low electron density are regarded as having reached convergence at the fifth SCF iteration.

In the example depicted in FIG. 8, the electron density difference at points in subspaces with medium electron density becomes less than or equal to the allowable value at the fifth SCF iteration. On the other hand, the difference change rate at the points in subspaces with medium electron density remains above the allowable value even at the seventh iteration. In this case, the points in subspaces with medium electron density are regarded as having reached convergence at the fifth SCF iteration.

In the example depicted in FIG. 8, the electron density difference at points in subspaces with high electron density becomes less than or equal to the allowable value at the fourth SCF iteration. On the other hand, the difference change rate at the points in subspaces with high electron remains above the allowable value even at the seventh iteration. In this case, the points in subspaces with high electron density are regarded as having reached convergence at the fourth SCF iteration.

In the example depicted in FIG. 8, the number of SCF iterations until the overall electron density converges is five, which is the maximum among the numbers of iterations in the subspaces with low (5), medium (5), and high (4) electron density. If the convergence criterion for the difference change rate is not used as a convergence criterion, the number of SCF iterations until the overall electron density converges becomes seven. That is, by applying the convergence criterion for the difference change rate in a logical disjunction, the number of SCF iterations is reduced.

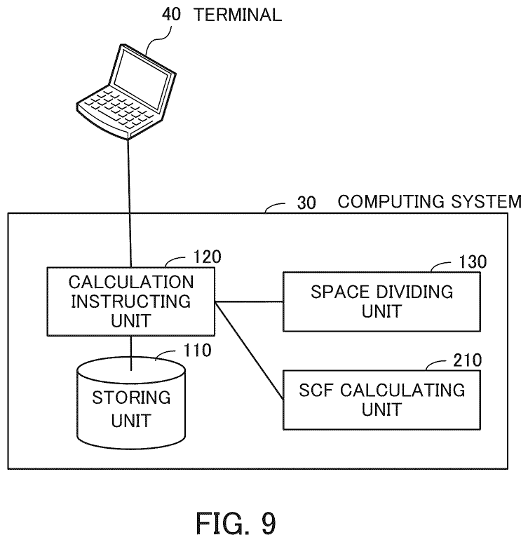

FIG. 9 is a block diagram illustrating an example functions implemented by the computing system. The of computing includes a storing unit 110, a calculation instructing unit 120, a space dividing unit 130, and an SCF calculating unit 210. The storing unit 110, the calculation instructing unit 120, and the space dividing unit 130 are, for example, functions implemented by the control node 100. The SCF calculating unit 210 is a function implemented, for example, by the computing nodes 200a, 200b, and the like.

The storing unit 110 stores information related to a substance to be analyzed, such as the atoms contained therein and known atomic configurations.

The calculation instructing unit 120, upon receiving an analysis instruction (e.g., to analyze physical properties) for a specified substance from the terminal 40, transmits a space division instruction to the space dividing unit 130 to perform analysis on that substance. The analysis instruction includes, for example, an initial value of the electron density for each point in the analysis space. The analysis instruction also includes, for example, convergence criteria, such as the allowable values for the difference and the difference change rate.

Upon receiving information from the space dividing unit 130 indicating the result of space division (e.g., a list of coordinates of points included in each subspace), the calculation instructing unit 120 transmits an instruction for electron density calculation, including subspace information, to the SCF calculating unit 210. Then, upon receiving the calculation result from the SCF calculating unit 210, the calculation instructing unit 120 transmits the result to the terminal 40.

The space dividing unit 130 divides the analysis space into subspaces with low electron density, medium electron density, and high electron density. For example, the space dividing unit 130 may divide the space based on the initial values of the electron density.

The SCF calculating unit 210 repeats the SCF calculation to obtain the electron density using DFT. During this process, the SCF calculating unit 210 determines whether the electron density has converged for each subspace. When the electron density in all subspaces has converged, the SCF calculating unit 210 transmits the electron density at each point at that time to the calculation instructing unit 120 as the calculation result.

The functions of the components illustrated in FIG. 9 may be implemented, for example, by executing program modules corresponding to those components on a computer. For example, the control node 100 executes programs corresponding to the calculation instructing unit 120 and the space dividing unit 130, thereby implementing the calculation instructing unit 120 and the space dividing unit 130. The calculation instructing unit 120 implemented on the control node 100 instructs one or more of the computing nodes 200a, 200b, and the like to execute jobs corresponding to the SCF calculating unit 210. The instructed computing nodes implement the SCF calculating unit 210 by executing the program corresponding to the specified job.

The following describes the procedure of an energy computation process for a substance using SCF calculation.

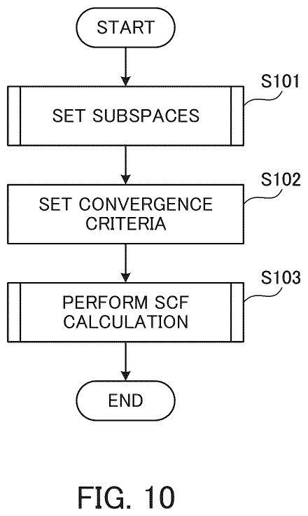

FIG. 10 is a flowchart illustrating an example of the procedure for the energy computation process. The following describes the steps illustrated in FIG. 10 in accordance with the step numbers.

[Step S101] First, the space dividing unit 130 divides the analysis space to set subspaces. For example, the calculation instructing unit 120, upon receiving an analysis instruction, transmits a space division instruction to the space dividing unit 130. In response to the space division instruction, the space dividing unit 130 performs a subspace setting process. Details of the subspace setting process will be described later.

[Step S102] Once the space dividing unit 130 completes the subspace setting process, the calculation instructing unit 120 sets convergence criteria. For example, the calculation instructing unit 120 sets, as the convergence criteria, allowable values for the difference and the difference change rate, as specified in the analysis instruction. The convergence criteria may be set, for example, for each subspace.

[Step S103] The SCF calculating unit 210 repeats the SCF calculation until the overall electron density converges. Details of the SCF calculation process will be described later.

In this manner, the computing system 30 performs SCF calculation after the analysis space is divided in advance into subspaces. In the spatial division into subspaces, it is determined, for each point that is a target of the electron density calculation, which subspace the point is to be included in.

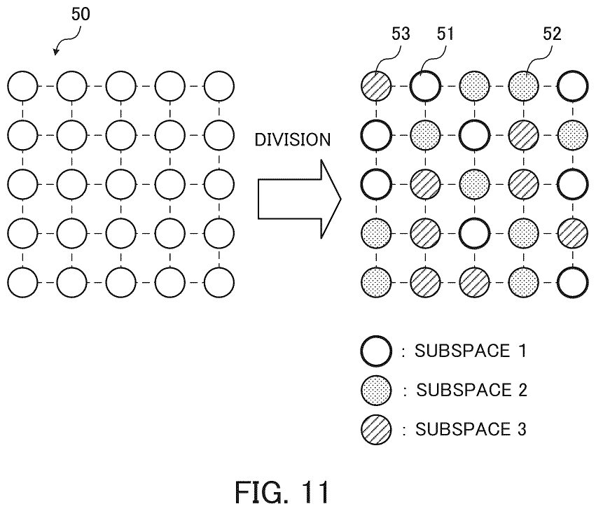

FIG. 11 illustrates an example of subspaces. An entire space 50 to be analyzed includes a set of multiple points. The space 50 is divided into two or more subspaces 51 to 53. Each of the subspaces 51 to 53 includes one or more points. The points included in a single subspace may be discontinuous. For example, the space dividing unit 130 determines, based on the initial values of the electron densities at the respective points, to which subspace each point belongs.

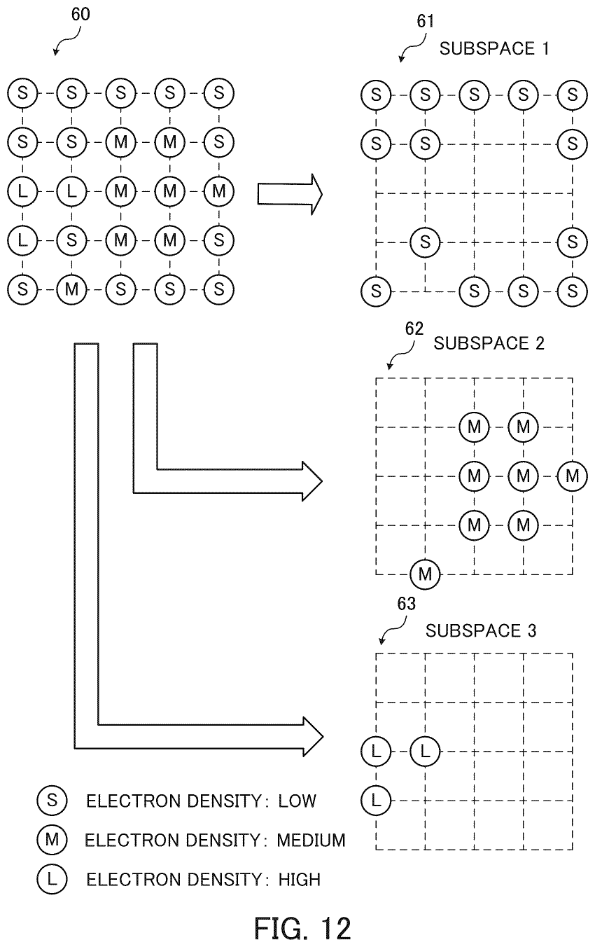

FIG. 12 illustrates an example of subspaces set based on electron densities. An entire space 60 includes points having a small initial value of electron density (denoted as “S” in FIG. 12), points having a medium initial value of electron density (denoted as “M”), and points having a large initial value of electron density (denoted as “L”).

The space dividing unit 130 groups the points based on the electron densities and sets each group as a subspace. For example, the space dividing unit 130 includes points having an initial value of electron density lower than a first threshold in a subspace 61 (i.e., “subspace 1” with low electron density, corresponding to “S” in FIG. 12). The space dividing unit 130 also includes the points having an initial value of electron density equal to or greater than the first threshold and lower than a second threshold in a subspace 62 (“subspace 2” with medium electron density, corresponding to “M”). The second threshold is greater than the first threshold. Further, the space dividing unit 130 includes points having an initial value of electron density equal to or greater than the second threshold in a subspace 63 (“subspace 3” with high electron density, corresponding to “L”).

In this manner, the points included in the entire space 60 are grouped, and each group is set as one of the subspaces 61 to 63.

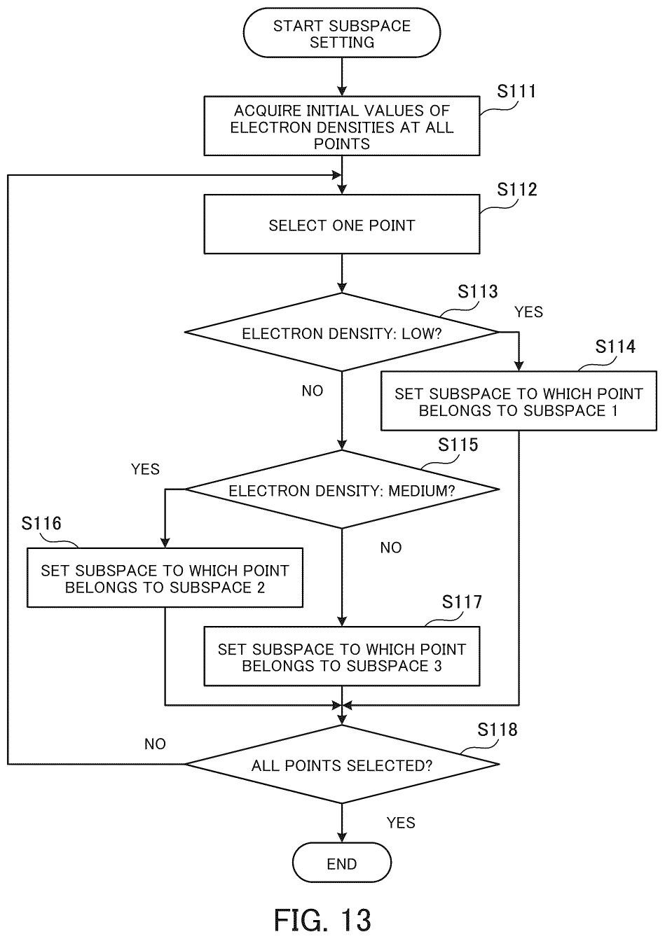

FIG. 13 is a flowchart illustrating an example of a procedure for a subspace setting process. The following describes the steps illustrated in FIG. 13 in accordance with the step numbers.

[Step S111] The space dividing unit 130 acquires the initial values of the electron densities at the respective points. For example, the space dividing unit 130 obtains the initial n densities specified in the analysis instruction from the calculation instructing unit 120.

[Step S112] The space dividing unit 130 selects one unselected point included in the entire space.

[Step S113] The space dividing unit 130 determines whether the electron density of the selected point is “low”. For example, if the electron density of the selected point is less than the first threshold, the space dividing unit 130 determines the electron density is as “low”. If the electron density is “low”, the space dividing unit 130 advances the process to step S114. If the electron density is not “low”, the space dividing unit 130 advances the process to step S115.

[Step S114] The space dividing unit 130 assigns the selected point to “subspace 1”. Then, the space dividing unit 130 advances the process to step S118.

[Step S115] The space dividing unit 130 determines whether the electron density of the selected point is “medium”. For example, if the electron density is equal to or greater than the first threshold and less than the second threshold, the space dividing unit 130 determines the electron density is as “medium”. If the electron density is “medium”, the space dividing unit 130 advances the process to step S116. If the electron density is not “medium”, the space dividing unit 130 advances the process to step S117.

[Step S116] The space dividing unit 130 assigns the selected point to “subspace 2”. Then, the space dividing unit 130 advances the process to step S118.

[Step S117] The space dividing unit 130 sets the subspace to which the selected point belongs to “subspace 3”.

[Step S118] The space dividing unit 130 determines whether all points have been selected. If all points have been selected, the space dividing unit 130 ends the subspace setting process. If there remain unselected points, the space dividing unit 130 returns the process to step S112.

In this manner, the entire space is divided into a plurality of subspaces. Once the subspaces are generated, the calculation instructing s convergence criteria for each subspace. The set convergence criteria are represented, for example, by allowable values for the difference and the difference change rate. These allowable values for the difference and the difference change rate are stored as allowable value information, for example, in the memory 102.

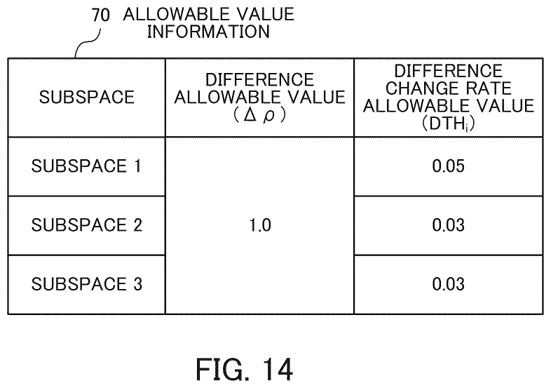

FIG. 14 illustrates an example of allowable value information. In allowable value information 70, for example, the difference allowable value (Δρ) and the difference change rate allowable value (DTHᵢ) are associated with the names of the respective subspaces. In the example of FIG. 14, the difference allowable values are set to the same value across all subspaces. However, different values may be assigned to each subspace.

The convergence criteria for each subspace are defined by the difference allowable value and the difference change rate allowable value. Once the convergence criteria are set, the SCF calculating unit 210 executes the SCF calculation.

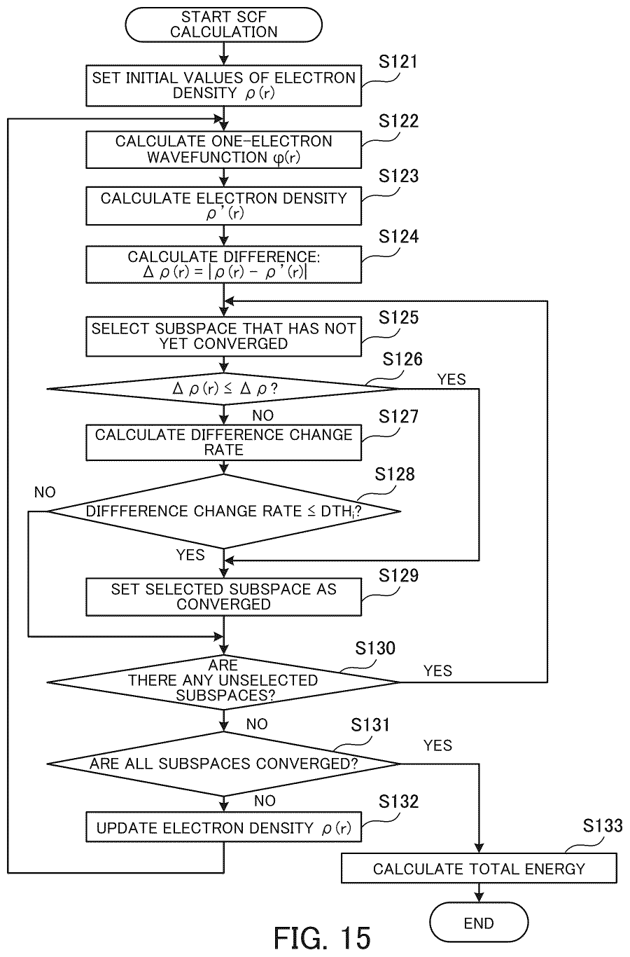

FIG. 15 is a flowchart illustrating an example of a procedure for the SCF calculation process. The following describes the steps illustrated in FIG. 15 in accordance with the step numbers.

[Step S121] The SCF calculating unit 210 sets the initial values of the electron density ρ(r), which are predetermined for all points indicated by position vectors r.

[Step S122] The SCF calculating unit 210 calculates a one-electron wavefunction (r) (represented as a single-stroke ϕ in FIG. 15). For example, the SCF calculating unit 210 may solve the Kohn-Sham equation to compute the one-electron wavefunction ϕ(r).

[Step S123] The SCF calculating unit 210 calculates the electron density ρ′(r) at each point based on the one-electron wavefunction ϕ (r).

[Step S124] The SCF calculating unit 210 calculates the difference Δρ(r) between the electron densities ρ(r) and ρ′(r) at each point. For example, the difference Δρ(r) may be calculated as: Δρ(r)=|ρ(r)−ρ′(r)|.

[Step S125] The SCF calculating unit 210 selects one of the subspaces that has not yet converged.

[Step S126] The SCF calculating unit 210 determines whether the difference Δρ(r) at all points in the selected subspace is less than or equal to the allowable value Δρ. For example, the SCF calculating unit 210 extracts the maximum value among the differences Δρ(r) for the respective points in the selected subspace. Then, the SCF calculating unit 210 determines that the differences Δρ(r) at all points in the selected subspace are less than or equal to the allowable value Δρ if the extracted maximum value is less than or equal to the allowable value Δρ.

If the SCF calculating unit 210 determines that the differences Δρ(r) at all points in the selected subspace are less than or equal to the allowable value Δρ, the SCF calculating unit 210 advances the process to step S129. If the difference Δρ(r) of at least one point in the selected subspace exceeds the allowable value Δρ, the SCF calculating unit 210 advances the process to step S127.

[Step S127] The SCF calculating unit 210 calculates the difference change rates for all points in the selected subspace.

[Step S128] The SCF calculating unit 210 determines whether the difference change rates at all points in the selected subspace are less than or equal to the allowable value DTHᵢ for that subspace. For example, the SCF calculating unit 210 extracts the maximum value among the difference change rates for the respective points in the selected subspace. Then, the SCF calculating unit 210 determines that the difference change rates at all points in the selected subspace are less than or equal to the allowable value DTHᵢ if the extracted maximum value is less than or equal to the allowable value DTHᵢ.

If the difference change rates at all points in the selected subspace are less than or equal to the allowable value DTHᵢ, the SCF calculating unit 210 advances the process to step S129. If at least one point in the selected subspace has a difference change rate that exceeds the allowable value DTHᵢ, the SCF calculating unit 210 advances the process to step S130.

[Step S129] The SCF calculating unit 210 sets the selected subspace as converged.

[Step S130] The SCF calculating unit 210 determines whether there remain any unselected subspaces among the subspaces that have not reached convergence. If there are unselected subspaces, the SCF calculating unit 210 returns the process to step S125. If all subspaces have been selected, the SCF calculating unit 210 advances the process to step S131.

[Step S131] The SCF calculating unit 210 determines whether all subspaces have converged. If all subspaces have converged, the SCF calculating unit 210 advances the process to step S133. If there remain any subspaces that have not reached convergence, the SCF calculating unit 210 advances the process to step S132.

[Step S132] The SCF calculating unit 210 updates the electron density ρ(r). For example, the SCF calculating unit 210 sets the electron density ρ′(r) as the electron density ρ(r). After updating the electron density ρ(r), the SCF calculating unit 210 returns the process to step S122.

[Step S133] The SCF calculating unit 210 calculates the total energy of the system based on the latest electron density ρ′(r) at each point.

In this manner, the SCF calculation is performed, and the total energy at the time the electron density has converged is obtained. By applying the SCF calculation depicted in FIG. 15 to a DFT calculation, the calculation is accelerated. The acceleration factor of the calculation may be obtained using the number of iterations at which the convergence criteria for the difference and the difference change rate are satisfied, based on the following expression: Acceleration factor=max(nd₁, nd₂, . . . , ndₙ)/max(min(nd₁, nr₁), min(nd₂, nr₂), . . . , min(ndₙ, nrₙ)).

Here, n is a natural number indicating the number of subspaces. nd₁, nd₂, . . . , and ndₙ respectively indicate the number of iterations in which the difference convergence criterion is satisfied in each of the first through n-th subspaces. nr₁, nr₂′, . . . , and nrₙ respectively indicate the number of iterations in which the difference change rate convergence criterion is satisfied in each of the first through n-th subspaces. max( ) indicates the maximum value among the numbers inside the parentheses. min( ) indicates the minimum value among the numbers inside the parentheses.

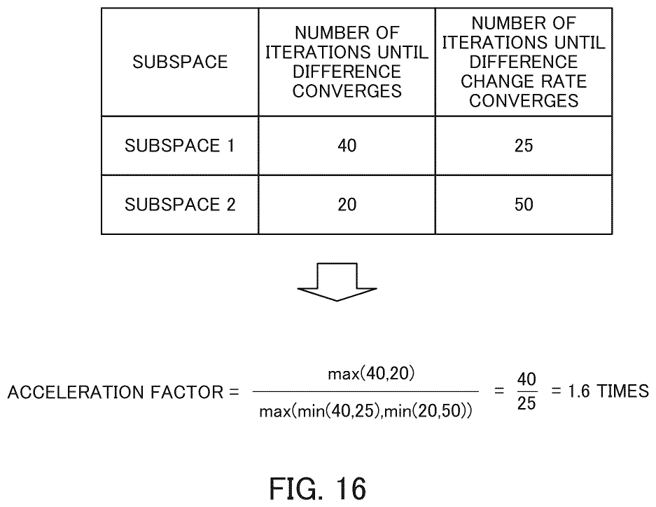

FIG. 16 illustrates an example of the acceleration factor. For example, consider a case with two subspaces. It is assumed that, in “subspace 1”, the difference converges (i.e., the convergence criterion is satisfied) at the fortieth SCF iteration, and the difference change rate converges at the twenty-fifth SCF iteration. It is also assumed that, in “subspace 2”, the difference converges at the twentieth SCF iteration, and the difference change rate converges at the fiftieth SCF iteration. In this case, the acceleration factor is calculated as follows: Acceleration factor=max(40, 20)/max(min(40, 25), min(20, 50))=40/25=1.6 times.

The acceleration of the SCF calculation described in the second embodiment is applicable, for example, to molecular dynamics computations using a program called CP2K. CP2K is an open-source first-principles calculation library that supports both the pseudopotential method and all-electron method. In CP2K, Gaussian basis sets, plane-wave basis sets, and mixed basis sets thereof are available as basis sets. CP2K also achieves favorable performance in large-scale parallel computations and linear-scaling calculations, and supports various first-principles calculation methods, including density functional theory and the Hartree-Fock method.

Even when there is only one subspace (i.e., no spatial division is performed), acceleration of SCF calculation is achieved by performing convergence determination based on the logical disjunction of the difference and the difference change rate.

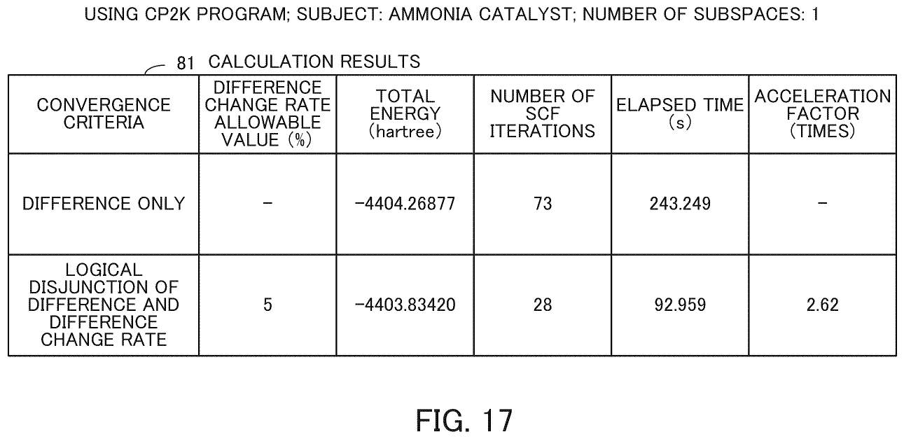

FIG. 17 illustrates an example of acceleration of the SCF calculation. FIG. 17 depicts calculation results 81 obtained when the structure of an ammonia catalyst is computed by DFT using a CP2K program. The number of subspaces is one. The SCF calculation is performed in two patterns: one using only the difference of the electron density as the convergence criterion, and the other using both the difference and the difference change rate of the electron density as the convergence criteria. When the difference change rate is used, the allowable value for the difference change rate is set to 5%.

When only the difference of the electron density is used as the convergence criterion, the total energy is −4404.26877, the number of SCF iterations is 73, and the elapsed time until convergence is 243.249 seconds. When both the difference and the difference change rate of the electron density are used as the convergence criteria, the total energy is −4403.83420, the number of SCF iterations is 28, the elapsed time until convergence is 92.959 seconds, and the acceleration factor is 2.62 times.

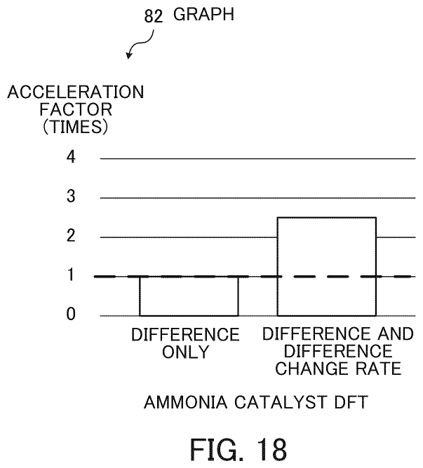

FIG. 18 illustrates the acceleration factor when both the difference and the difference change rate of the electron density are used as convergence criteria. A graph 82 illustrates the acceleration factors in two cases: one where only the difference of the electron density is used as the convergence criterion, and the other where both the difference and the difference change rate of the electron density are used. In the case of using only the difference, the acceleration factor is 1. On the other hand, when both the difference and the difference change rate are used, the acceleration factor is 2.62.

As described above, using both the difference and the difference change rate of the electron density for convergence determination leads to a faster computation. Moreover, as depicted in FIG. 17, there is no significant difference in the obtained total energy. In other words, the computation time is reduced without compromising computational accuracy.

By applying the technique described in the second embodiment, for example, it becomes possible to efficiently carry out a wide-range exploration service for new material that integrates HPC and artificial intelligence (AI).

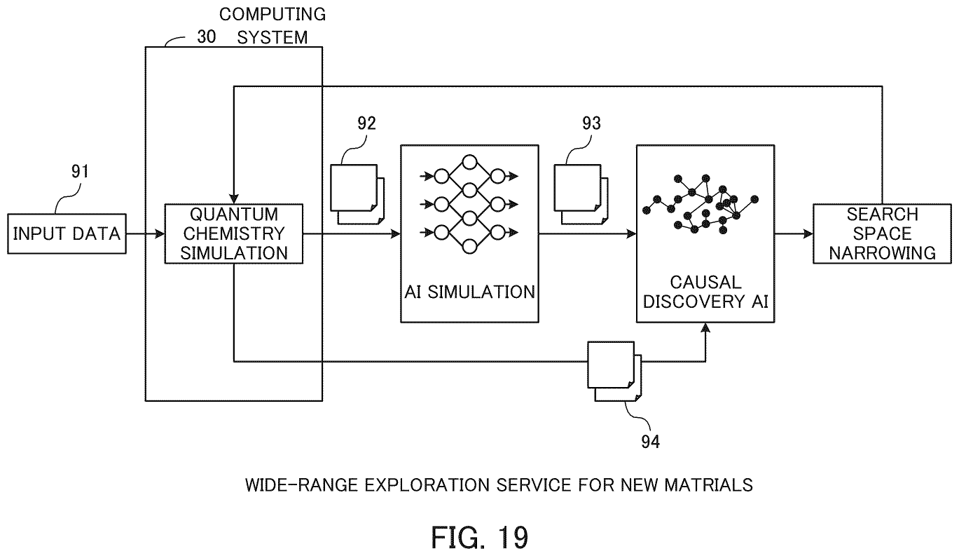

FIG. 19 illustrates an example of a wide-range exploration service. For example, the computing system 30 acquires input data 91 from a user, the data indicating initial conditions related to a substance such as a material to be explored. The computing system 30 performs quantum chemistry simulation at high speed based on the input data 91. By employing the technique described in the second embodiment, the computing system 30 is able to perform high-accuracy simulations approximately ten times faster. As a result, the computing system 30 is able to generate high-quality data 92 and 94 related to material structures.

The data 92 generated by the computing system 30 is analyzed, for example, through AI simulation based on technologies such as Graph Neural Network (GNN), to evaluate material properties and other characteristics. By using data 93 obtained from the AI simulation together with the data 94 generated by the computing system 30, causal relationships, such as those between numerous material candidates and their properties, are analyzed through causal discovery AI, even in cases where such analysis would be infeasible for humans. This process enables the discovery of new findings and insights through causal analysis.

The results of the causal relationship analysis are reflected, for example, in the search conditions for the quantum chemistry simulation, and are used to narrow the search space. As the search space is appropriately narrowed, for example, a substance with properties desired by the user is identified at an earlier stage.

### (c) Other Embodiments

In the second embodiment, the disclosed technique is illustrated through an example involving a wide-ranging new material exploration service using AI. Nevertheless, the technique is also applicable to other types of information processing services.

In the second embodiment, DFT calculations are performed using HPC. However, if the number of atoms in the target substance is small, the DFT calculation described in the second embodiment may also be performed using a non-HPC computer.

According to one aspect, the computation time for the electron density is reduced.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable recording medium storing therein a computer program that causes a computer to execute a process comprising:
iteratively calculating, using a self-consistent field method, an electron density at each of a plurality of points in a space where a substance exists;
determining, for each of the plurality of points, each time the electron density is calculated, whether a plurality of indicator values based on the calculated electron density satisfies respective convergence criteria respectively associated with the plurality of indicator values; and
terminating, for each of the plurality of points, the iteratively calculating of the electron density upon at least one of the plurality of indicator values satisfying the associated convergence criterion.

2. The non-transitory computer-readable recording medium according to claim 1, wherein
the process further includes:
classifying each of the plurality of points into one of a plurality of subspaces based on an initial value of the electron density at the respective point, and
the determining of whether the respective convergence criteria are satisfied includes:
calculating a plurality of first indicator values for a first determination target point that belongs to a first subspace among the plurality of subspaces, and
determining, based on the respective convergence criteria respectively associated with the plurality of indicator values, the respective convergence criteria being set for each of the plurality of subspaces, whether each of the plurality of first indicator values calculated satisfies the associated convergence criterion set for the first subspace to which the first determination target point belongs.

3. The non-transitory computer-readable recording medium according to claim 2, wherein the determining of whether the respective convergence criteria are satisfied includes:
determining that the first subspace among the plurality of subspaces has converged upon at least one of the plurality of indicator values calculated for each point belonging to the first subspace satisfying the associated convergence criterion set for the first subspace, and
determining, for each point belonging to a second subspace other than the first subspace determined to have converged, whether each of the plurality of indicator values satisfies the associated convergence criterion set for the second subspace.

4. The non-transitory computer-readable recording medium according to claim 1, wherein the plurality of indicator values includes a difference between a previous calculation result of the electron density and a current calculation result of the electron density, and a difference change rate indicating a degree of change between a previous calculation result of the difference of the electron density and a current calculation result of the difference of the electron density.

5. A quantum chemistry computation method comprising:
iteratively calculating, by a processor, using a self-consistent field method, an electron density at each of a plurality of points in a space where a substance exists;
determining, by the processor, each of the plurality of points, each time the electron density is calculated, whether a plurality of indicator values based on the calculated electron density satisfies respective convergence criteria respectively associated with the plurality of indicator values; and
terminating, by the processor, for each of the plurality of points, the iteratively calculating of the electron density upon at least one of the plurality of indicator values satisfying the associated convergence criterion.

6. The quantum chemistry computation method according to claim 5, further comprising classifying, by the processor, each of the plurality of points into one of a plurality of subspaces based on an initial value of the electron density at the respective point,
wherein the determining of whether the respective convergence criteria are satisfied includes:
calculating a plurality of first indicator values for a first determination target point that belongs to a first subspace among the plurality of subspaces, and
determining, based on the respective convergence criteria respectively associated with the plurality of indicator values, the respective convergence criteria being set for each of the plurality of subspaces, whether each of the plurality of first indicator values calculated satisfies the associated convergence criterion set for the first subspace to which the first determination target point belongs.

7. The quantum chemistry computation method according to claim 6, wherein the determining of whether the respective convergence criteria are satisfied includes:
determining that the first subspace among the plurality of subspaces has converged upon at least one of the plurality of indicator values calculated for each point belonging to the first subspace satisfying the associated convergence criterion set for the first subspace; and
determining, for each point belonging to a second subspace other than the first subspace determined to have converged, whether each of the plurality of indicator values satisfies the associated convergence criterion set for the second subspace.

8. The quantum chemistry computation method according to claim 5, wherein the plurality of indicator values includes a difference between a previous calculation result of the electron density and a current calculation result of the electron density, and a difference change rate indicating a degree of change between a previous calculation result of the difference of the electron density and a current calculation result of the difference of the electron density.

9. An information processing apparatus comprising:
a memory; and
a processor coupled to the memory and the processor configured to:
iteratively calculate, using a self-consistent field method, an electron density at each of a plurality of points in a space where a substance exists,
determine, for each of the plurality of points, each time the electron density is calculated, whether a plurality of indicator values based on the calculated electron density satisfies respective convergence criteria respectively associated with the plurality of indicator values, and
terminate, for each of the plurality of points, the iteratively calculating of the electron density upon at least one of the plurality of indicator values satisfying the associated convergence criterion.

10. The information processing apparatus according to claim 9, wherein:
the processor is further configured to classify each of the plurality of points into one of a plurality of subspaces based on an initial value of the electron density at the respective point, and
in determining whether the respective convergence criteria are satisfied, the processor is configured to:
calculate a plurality of first indicator values for a first determination target point that belongs to a first subspace among the plurality of subspaces, and
determine, based on the respective convergence criteria respectively associated with the plurality of indicator values, the respective convergence criteria being set for each of the plurality of subspaces, whether each of the plurality of first indicator values calculated satisfies the associated convergence criterion set for the first subspace to which the first determination target point belongs.

11. The information processing apparatus according to claim 10, wherein in determining whether the respective convergence criteria are satisfied, the processor is configured to determine that the first subspace among the plurality of subspaces has converged upon at least one of the plurality of indicator values calculated for each point belonging to the first subspace satisfying the associated convergence criterion set for the first subspace, and determine, for each point belonging to a second subspace other than the first subspace determined to have converged, whether each of the plurality of indicator values satisfies the associated convergence criterion set for the second subspace.

12. The information processing apparatus according to claim 9, wherein the plurality of indicator values includes a difference between a previous calculation result of the electron density and a current calculation result of the electron density, and a difference change rate indicating a degree of change between a previous calculation result of the difference of the electron density and a current calculation result of the difference of the electron density.
